(19) United States
(12) Patent Application Publication
(10) Pub. No.: US 2008/0306349 A1
(43) Pub. Date: Dec. 11, 2008

(54) Visual Working Memory Assessment Using Abstract Symbols and Associated Methods

(75) Inventors: Lisa W. Drozdick, San Antonio, TX (US); James A. Holdnack, Bear, DE (US); Mary Diane Sichi, San Antonio, TX (US); Jayme LeRae Lyon, San Antonio, TX (US)

Correspondence Address: JACQUELINE E. HARTT, PH.D;Allen Dyer Doppelt Milbrath & Gilchrist, P.A., 255 S. Orange Ave., Suite 1401, P.O. Box 3791, ORLANDO, FL, 32801-3791, US

(73) Assignee: HARCOURT ASSESSMENT, INC., San Antonio, TX (US)

(21) Appl. No.: 11/760,154

(22) Filed: Jun. 8, 2007

(51) Int. Cl. A61B 5/00

(52) U.S. Cl. 600/300

(57) ABSTRACT

A neuropsychological assessment of visual working memory includes a first display having a first set of distinct abstract elements positioned in a first order. A second display includes a second set of distinct abstract symbols positioned in a second order. The second set includes all the abstract elements of the first set plus at least one additional distinct abstract element not present in the first set. The first display is shown to an examinee. The examinee is asked to view the first display and to note the abstract elements in the first set and the first order thereof. The examinee is instructed to view the second display and to indicate, with reference to the second set, a location of each of the abstract elements present in the first set, in the first order.

### FIELD OF THE INVENTION

The present invention generally relates to neuropsychological assessments, and, in particular, to systems and methods for assessing spatial working memory.

### BACKGROUND

Working memory is defined here as the capacity to “hold” and mentally “manipulate” information in conscious awareness. The purpose of working memory is to enable a person to operate on a stimulus to solve a particular problem and then to forget that information. Examples of working memory include the ability to remember a telephone number, perform math in the mind, and write something down while listening (i.e., multi-tasking).

Auditory working memory measures are known in the art; however, it has been more problematic to assess the visual working memory system. In part, the difficulty lies in finding visual information that cannot be codified by the verbal memory system in a way that facilitates recall and to find a visual test that requires mental manipulation. Mental manipulation is considered an essential component of working memory.

It has also been difficult to create a non-computerized assessment of visual working memory owing to the need to present items visually with no auditory content within the limitations of a paper or stimulus book format.

Prior known assessments can be difficult to track and score if motor manipulation is required of the examinee.

Therefore, it would be desirable to provide a visual working memory assessment and method of creating and administering same. It would also be desirable to provide such an assessment that is easier for examiners to track and score.

### SUMMARY OF THE INVENTION

One aspect of the present invention is directed to a neuropsychological assessment of visual working memory. The assessment requires mental manipulation of visual information using a resequencing paradigm common to auditory working memory tasks. The assessment enables clinicians to evaluate visual resquencing without relying on highly symbolic (i.e., verbally encodable numbers or letters) information.

The neuropsychological assessment of visual working memory comprises a first display that comprises a first set of distinct abstract elements positioned in a first order. A second display comprises a second set of distinct abstract symbols positioned in a second order. The second set includes all the abstract elements of the first set plus at least one additional distinct abstract element not present in the first set.

Means are provided for displaying the first display to an examinee. Means are also provided for asking the examinee to view the first display and to note the abstract elements in the first set and the first order thereof. Means are further provided for instructing the examinee to view the second display and to indicate, with reference to the second set, a location of each of the abstract elements present in the first set, in the first order.

The features that characterize the invention, both as to organization and method of operation, together with further objects and advantages thereof, will be better understood from the following description used in conjunction with the accompanying drawing. It is to be expressly understood that the drawing is for the purpose of illustration and description and is not intended as a definition of the limits of the invention. These and other objects attained, and advantages offered, by the present invention will become more fully apparent as the description that now follows is read in conjunction with the accompanying drawing.

### BRIEF DESCRIPTION OF THE DRAWING

FIG. 1 is an exemplary first display for a first item of the present invention.

FIG. 2 is an exemplary second display for the first item.

FIG. 3 is a flowchart of an exemplary method of the present invention.

FIG. 4 is an exemplary first display for a second item

FIG. 5 is an exemplary second display for the second item.

FIG. 6 is an exemplary first display for a third item

FIG. 7 is an exemplary second display for the third item.

FIG. 8 is an exemplary first display for a fourth item

FIG. 9 is an exemplary second display for the fourth item.

### DETAILED DESCRIPTION OF THE PREFERRED EMBODIMENT

A description of the preferred embodiments of the present invention will now be presented with reference to FIGS. 1-9.

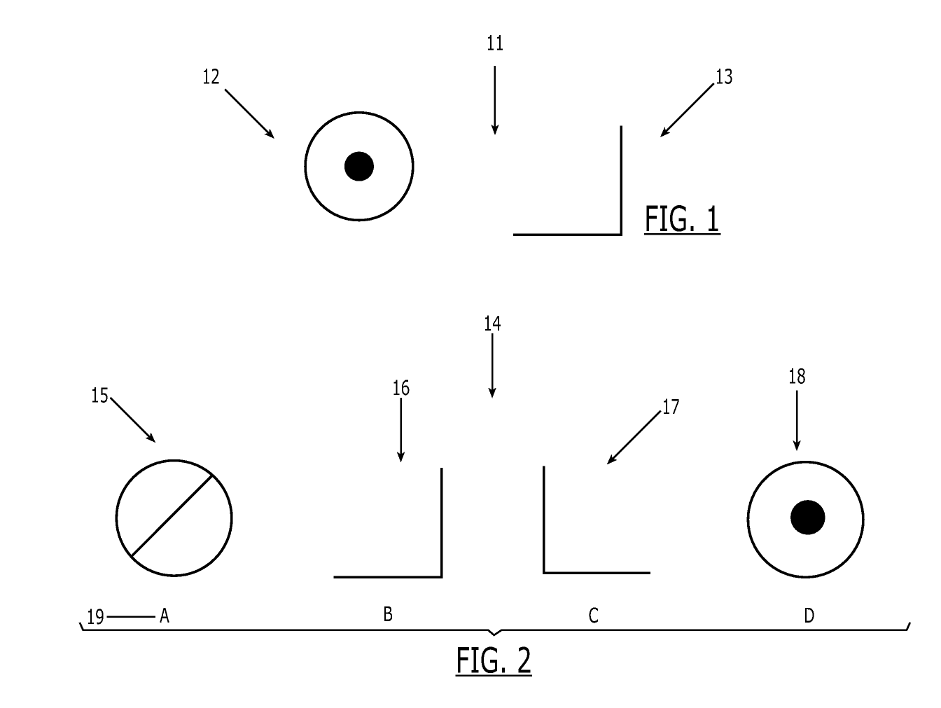

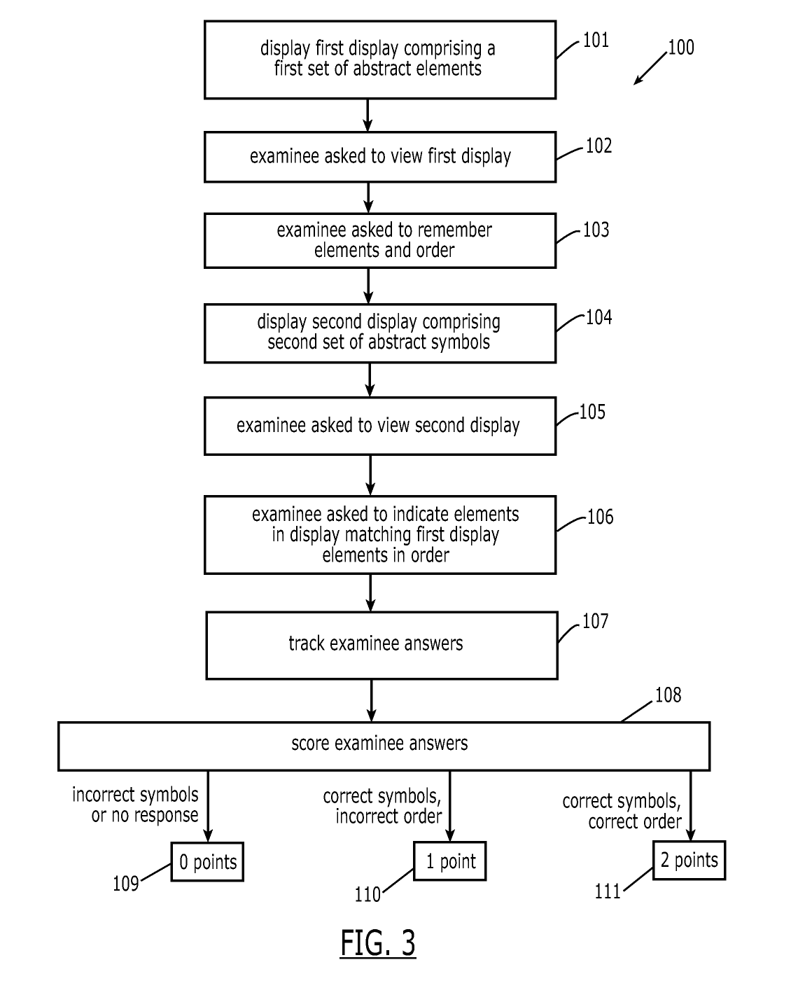

One aspect of the present invention is directed to a neuropsychological assessment and methods for delivering and scoring same. The method, using the assessment of the present invention, will first be described with a particular example illustrated in FIGS. 1 and 2, which is not intended as a limitation. The method 100, outlined in the flowchart of FIG. 3, comprises the step of displaying to an examinee a first display 11 (FIG. 1), which comprises a first set of distinct abstract elements 12,13 positioned in a first order (block 101). The examinee is shown the first display 11 for a predetermined period of time, for example, 5 seconds, although this is not intended as a limitation.

The examinee is asked to view the first display 11 (block 102) and to remember the abstract elements in the first set and the first order thereof (block 103).

A second display 14 (FIG. 2) is then shown to the examinee (block 104). The second display 14 comprises a second set of distinct abstract symbols 15-18 positioned in a second order. The second set 14 includes all the abstract elements 12,13 of the first set 11 plus at least one additional distinct abstract element not present in the first set 11. The second display 14 can further comprise an indicium 19 adjacent each of the abstract elements 15-18 of the second set 14.

The examinee is instructed to view the second display (block 105). The examinee is also asked to indicate, with reference to the second set 14, a location of each of the abstract elements present in the first set 11, in the first order, here elements 18,16 (block 106). The examinee can be instructed to indicate the location of each of the abstract elements by indicating the indicia 19 associated with the abstract elements, here, “D” and “B.” The examinee can also be asked to point at the elements from the first set 11.

The examinee indications can be tracked (block 107) and scored (block 108), for example, by awarding points based upon the examinee's answers. No points are awarded if the examinee chooses incorrect symbols or does not respond (block 109). One point is awarded if the correct symbols are chosen, but in the wrong order (block 110). Two points are awarded if the correct symbols are chosen, and in the correct order (block 111).

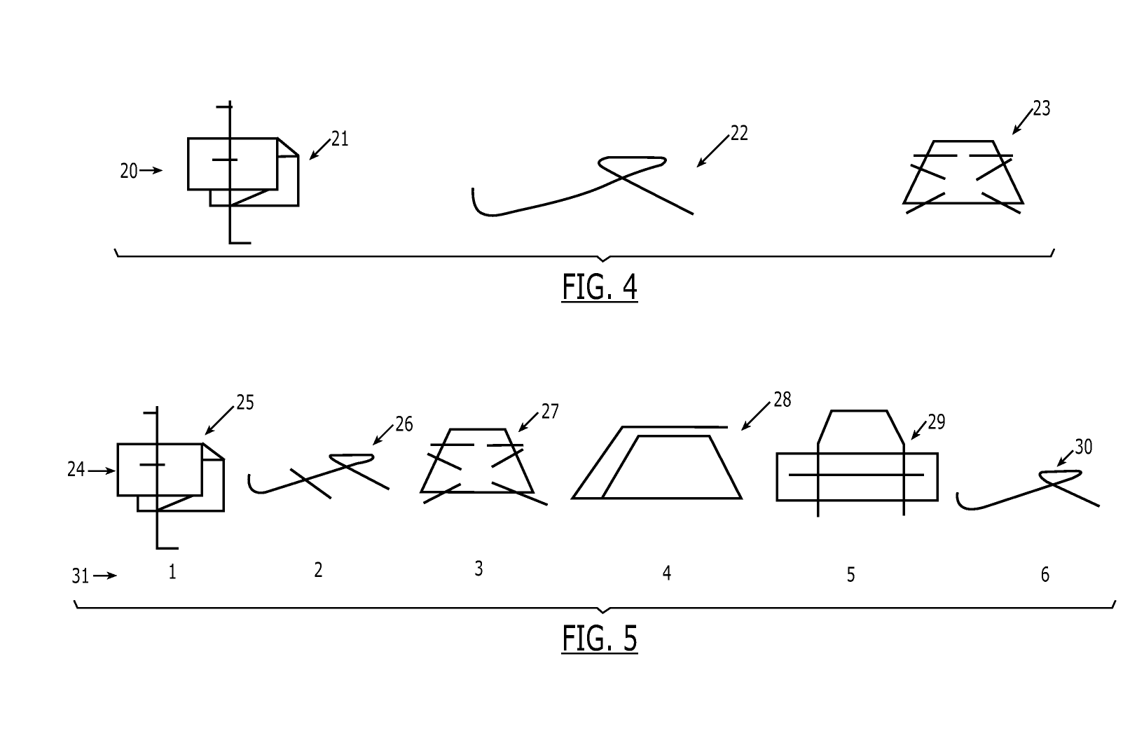

Three additional examples of items are illustrated in FIGS. 4-9. The first display 20 of FIG. 4 has three symbols 21-23 in a first order. The second display 24 of FIG. 5 has six symbols 25-30 in a second order, with numerical indicia 31. The examinee should name the second display symbols in the order 25,30,27.

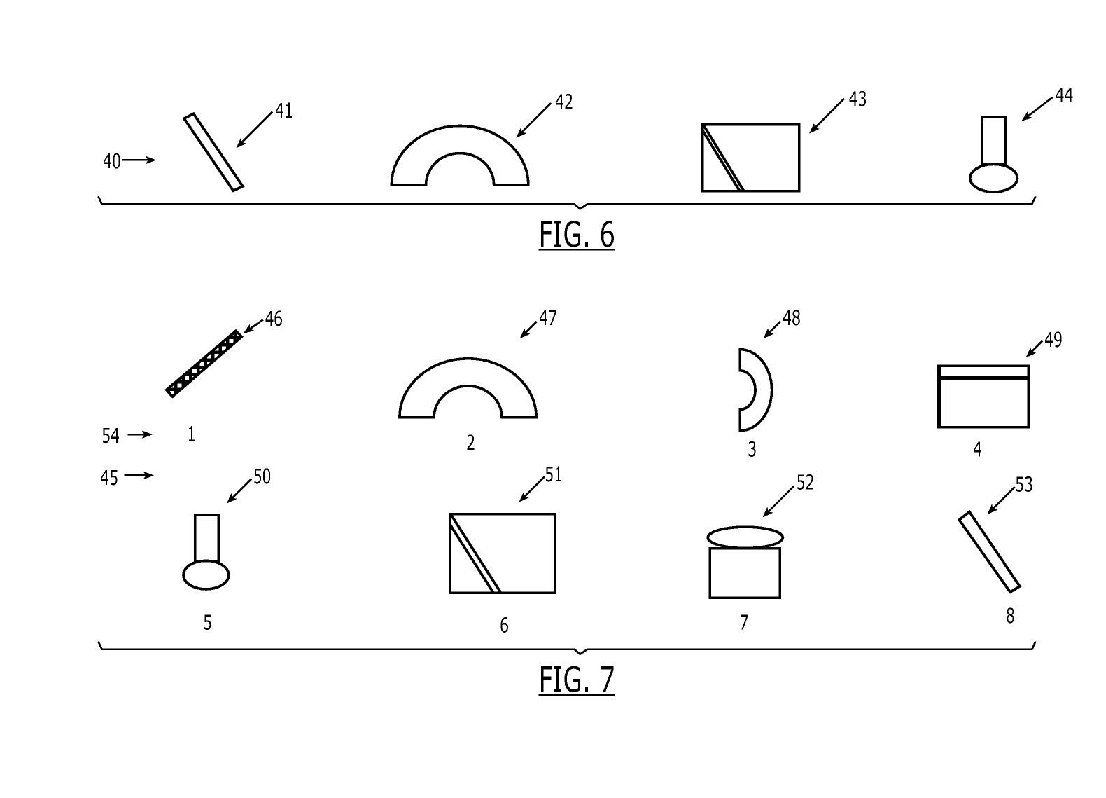

The first display 40 of FIG. 6 has four symbols 41-44 in a first order. The second display 45 of FIG. 7 has eight symbols 46-53 in a second order, with numerical indicia 54. The examinee should name the second display symbols in the order 53,47,51,50.

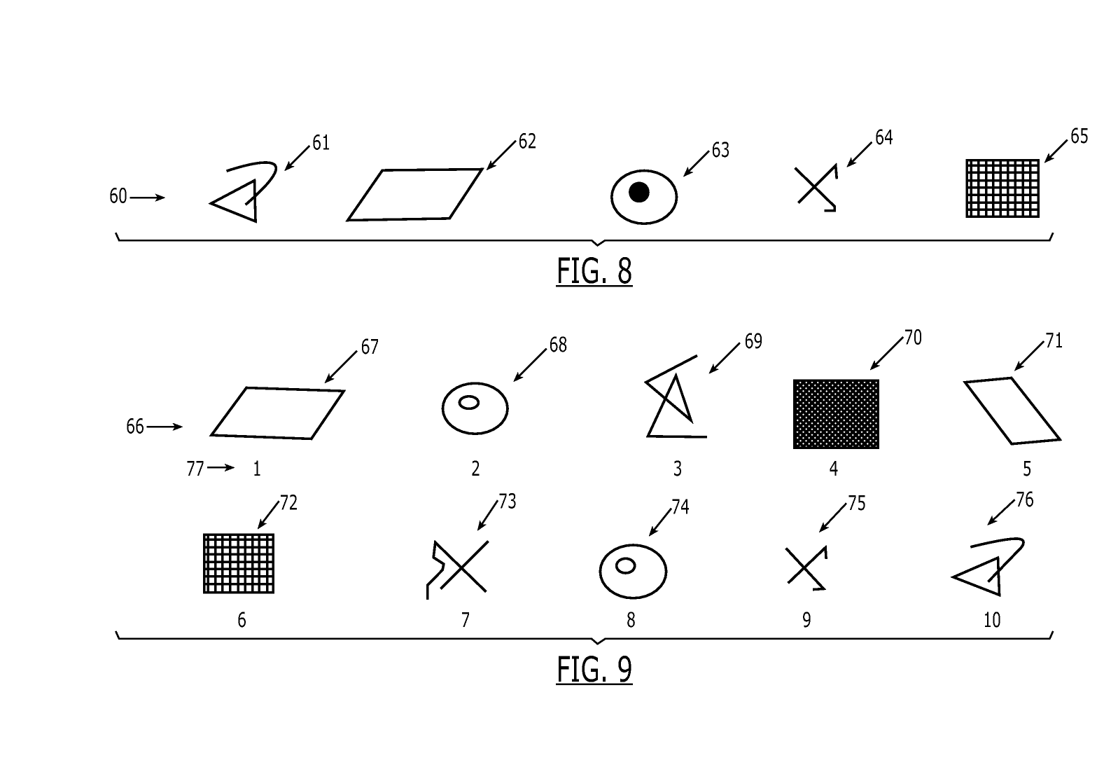

The first display 60 of FIG. 8 has five symbols 61-65 in a first order. The second display 66 of FIG. 9 has ten symbols 67-76 in a second order, with numerical indicia 77. The examinee should name the second display symbols in the order 76,67,74,75,72.

It will be understood by one of skill in the art that the invention can be practiced in a number of ways, including with the use of a stimulus book such as known in the art, other types of hard-copy displays, and computer displays with or without an interactive feature such as a touch screen.

In the foregoing description, certain terms have been used for brevity, clarity, and understanding, but no unnecessary limitations are to be implied therefrom beyond the requirements of the prior art, because such words are used for description purposes herein and are intended to be broadly construed. Moreover, the embodiments of the assessment illustrated and described herein are by way of example, and the scope of the invention is not limited to the exact details.

Having now described the invention, the operation and use of preferred embodiments thereof, and the advantageous new and useful results obtained thereby, the new and useful constructions, and reasonable equivalents thereof obvious to those skilled in the art, are set forth in the appended claims.

What is claimed is:

1. A neuropsychological assessment of visual working memory comprising:
a first display comprising a first set of distinct abstract elements positioned in a first order;
a second display comprising a second set of distinct abstract symbols positioned in a second order, the second set including all the abstract elements of the first set plus at least one additional distinct abstract element not present in the first set;
means for displaying the first display to an examinee;
means for asking the examinee to view the first display and to remember the abstract elements in the first set and the first order thereof; and
means for instructing the examinee to view the second display and to indicate, with reference to the second set, a location of each of the abstract elements present in the first set, in the first order.

2. The assessment recited in claim 1, wherein the second display further comprises an indicium adjacent each of the abstract elements of the second set.

3. The assessment recited in claim 2, wherein the instructing means comprises means for instructing the examinee to indicate the location of each of the abstract elements present in the first set, in the first order, by indicating the indicia associated with the abstract elements.

4. The assessment recited in claim 1, wherein the displaying means comprises means for displaying the first display for a predetermined period of time.

5. The assessment recited in claim 4, wherein the predetermined time comprises approximately 5 seconds.

6. The assessment recited in claim 1, wherein the means for instructing the examinee to indicate the location of each of the abstract elements present in the first set, in the first order, comprises means for instructing the examinee to point at each of the abstract elements present in the first set, in the first order.

7. The assessment recited in claim 1, further comprising means for tracking the examinee indications and for scoring the examinee indications.

8. A method of assessing visual working memory of an examinee comprising the steps of:
displaying to an examinee a first display comprising a first set of distinct abstract elements positioned in a first order;
displaying to the examinee a second display comprising a second set of distinct abstract symbols positioned in a second order, the second set including all the abstract elements of the first set plus at least one additional distinct abstract element not present in the first set;
asking the examinee to view the first display and to note the abstract elements in the first set and the first order thereof; and
instructing the examinee to view the second display and to indicate, with reference to the second set, a location of each of the abstract elements present in the first set, in the first order.

9. The method recited in claim 8, wherein the second display further comprises an indicium adjacent each of the abstract elements of the second set.

10. The method recited in claim 9, wherein the instructing step comprises means instructing the examinee to indicate the location of each of the abstract elements present in the first set, in the first order, by indicating the indicia associated with the abstract elements.

11. The method recited in claim 8, wherein the displaying step is carried out for a predetermined period of time.

12. The method recited in claim 11, wherein the predetermined time comprises approximately 5 seconds.

13. The method recited in claim 8, wherein the instructing step comprises instructing the examinee to point at each of the abstract elements present in the first set, in the first order.

14. The method recited in claim 8, further comprising the step of tracking the examinee indications and scoring the examinee indications.
